(19) United States
(12) Patent Application Publication
(10) Pub. No.: US 2005/0256416 A1
(43) Pub. Date: Nov. 17, 2005

(54) Heart rate detecting and displaying device

(76) Inventor: Yu Yu Chen, Chong Ho City (TW)

Correspondence Address: Yu Yu Chen, P.O.Box 10-69, Chong Ho, Taipei, 235, TW

(21) Appl. No.: 10/845,343

(22) Filed: May 12, 2004

(51) Int. Cl. A61B 5/0402

(52) U.S. Cl. 600/509

(57) ABSTRACT

A heart rate device includes a watch member having an area to show digit number of heart rates of users, and having another area to show status of the heart rates with different colors. An attaching device may be used to attach the watch member to the users, and may include a fastening belt for attaching to the users, and a securing device for securing the watch member to the fastening belt with such as hook and loop fastening members. The watch member may include a further area having a number of light members to show incoming phone. The watch member may further include two or more pointers to show time.

### BACKGROUND OF THE INVENTION

1. Field of the Invention

The present invention relates to a heart rate detecting and displaying device, and more particularly to a heart rate detecting and displaying device for attaching to various portions of users, and having a displaying area to show different heart rates with different colors.

2. Description of the Prior Art

Typical heart rate detecting or displaying devices have been developed for measuring or detecting heart rates of users, and normally comprise a screen to show or to display the heart rate patterns.

For example, U.S. Pat. No. 3,215,136 to Holter et al. discloses one of the typical electrocardiographic means comprising a screen to show or to display the heart rate patterns. However, the typical electrocardiographic means normally include a large volume that may not be carried with the users, and may not be easily attached to the users to measure or detect heart rates of users.

U.S. Pat. No. 6,033,344 to Trulaske et al. discloses a typical fitness apparatus having a heart rate control system to measure or detect heart rates of users, and comprising a pulse detection circuitry for attaching to chest portions of users with a chest belt.

However, the pulse detection circuitry of the typical heart rate control system may be attached to the chest portions of the users to detect the heart rate of the users, and the results of the heart rate are required to be sent to a treadmill display, and to show or to display the heart rate on the treadmill display.

The pulse detection circuitry of the typical heart rate control system and the treadmill display are separated from each other, and may not be easily carried with the users, and may not be easily attached to the users to measure or detect heart rates of users.

The present invention has arisen to mitigate and/or obviate the afore-described disadvantages of the conventional heart rate detecting and displaying devices.

### SUMMARY OF THE INVENTION

The primary objective of the present invention is to provide a heart rate detecting and displaying device for attaching to various portions of users.

The other objective of the present invention is to provide a heart rate detecting and displaying device including a displaying area to show different heart rates with different colors, for allowing the users to easily view the changing of the heart rate.

In accordance with one aspect of the invention, there is provided a heart rate device comprising a watch member including a first area to show digit number of heart rates of users, the watch member including a second area to show status of the heart rates with different colors, and attaching device for attaching the watch member to the users.

The attaching device includes a fastening belt for attaching to the users, and securing device for securing the watch member to the fastening belt. The securing device includes a first fastening member attached to the watch member, and a second fastening member attached to the fastening belt and engageable with the first fastening member, to secure the watch member to the fastening belt. The first and the second fastening members are preferably the hook and loop fastening members.

The securing device includes at least one pivot pin to secure the watch member to the fastening belt. The fastening belt may further include a first fastening member and a second fastening member attached thereto and engageable with each other, to attach the fastening belt and thus the watch member to the users.

The watch member may further include a third area having a plurality of light members disposed therein. The watch member may further include at least two pointers to show time.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a partial exploded view of a heart rate detecting and displaying device in accordance with the present invention;

FIG. 2 is a partial perspective view illustrating an attachment of the heart rate detecting and displaying device to arm of user;

FIG. 3 is a bottom plan view of the heart rate detecting and displaying device;

FIG. 4 is a top plan view of the heart rate detecting and displaying device, illustrating the operation of the heart rate detecting and displaying device;

FIG. 5 is a partial exploded view illustrating another arrangement of the heart rate detecting and displaying device;

FIG. 6 is a bottom plan view of the heart rate detecting and displaying device as shown in FIG. 5;

FIGS. 7, 8, 9 are partial perspective views illustrating attachments of the heart rate detecting and displaying device as shown in FIGS. 5 and 6 to various portions of users;

FIG. 10 is a partial perspective view illustrating an attachment of the heart rate detecting and displaying device to neck of user;

FIG. 11 is an enlarged partial perspective view illustrating the attachment of the heart rate detecting and displaying device to the neck of the user;

FIGS. 12, 13 are enlarged partial plan views illustrating the operation of the heart rate detecting and displaying device;

FIGS. 14, 15, 16 are plan schematic views illustrating the attachment of the heart rate detecting and displaying device to fastening belt; and

FIG. 17 is a partial perspective view illustrating the attachment of the heart rate detecting and displaying device to the wrist portion of the user.

### DETAILED DESCRIPTION OF THE PREFERRED EMBODIMENT

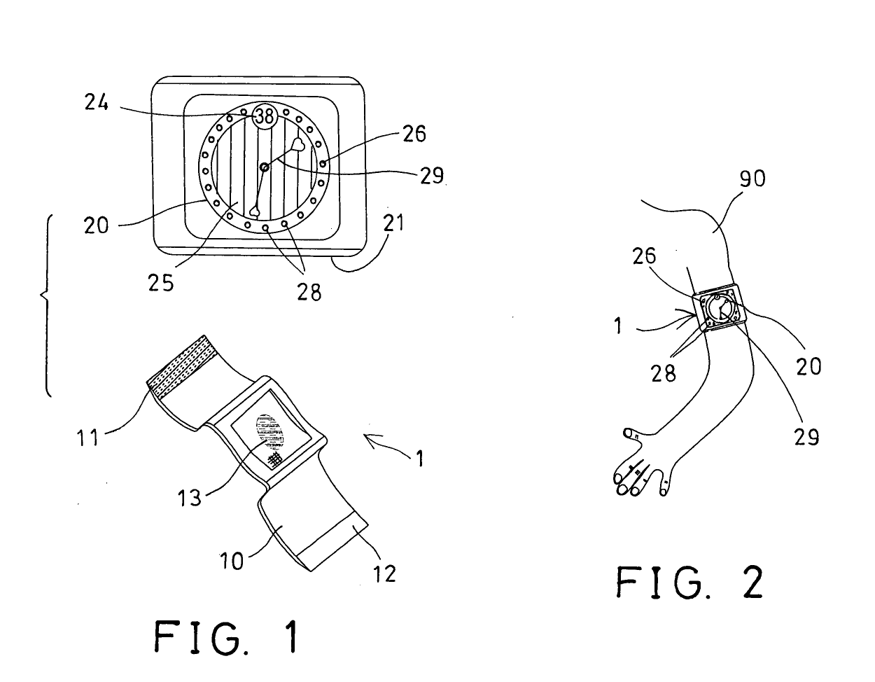

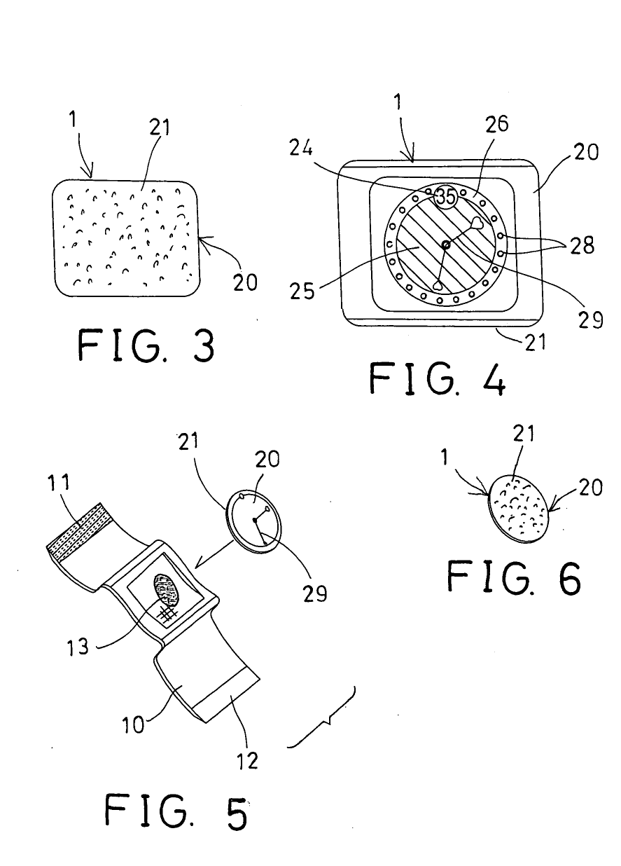

Referring to the drawings, and initially to FIGS. 1-3, a heart rate device or a heart rate detecting and displaying device 1 in accordance with the present invention comprises a fastening belt 10 including one or more fastening members 11, 12, such as hook and loop fastening members 11, 12 attached thereon.

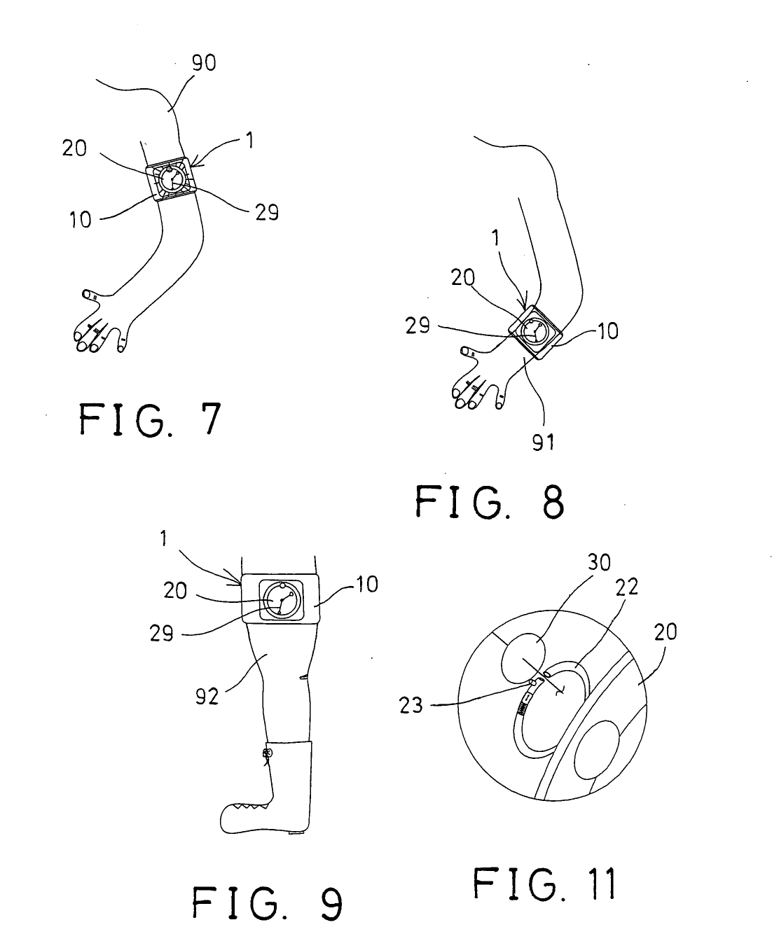

For example, the hook and loop fastening members 11, 12 may be engaged with each other, for attaching the fastening belt 10 to various portions of users, such as the arms 90 (FIGS. 2, 7), the wrist portion 91 (FIG. 8), the inner portion of the leg 92 (FIG. 9), or the like. The fastening belt 10 includes another fastening member 13, such as hook and loop fastening member 13 attached thereon.

The heart rate detecting and displaying device 1 further includes a body, such as a watch member 20 having another fastening member 21, such as hook and loop fastening member 21 attached thereon, for engaging with the fastening member 13 of the fastening belt 10 (FIGS. 1, 5), and thus for detachably attaching the watch member 20 to the fastening belt 10.

The watch member 20 also includes a pulse detection circuitry (not shown) to detect the heart rate of the users. One example of the pulse detection circuitry has been disclosed in U.S. Pat. No. 6,033,344 to Trulaske et al. which may be taken as a reference for the present invention.

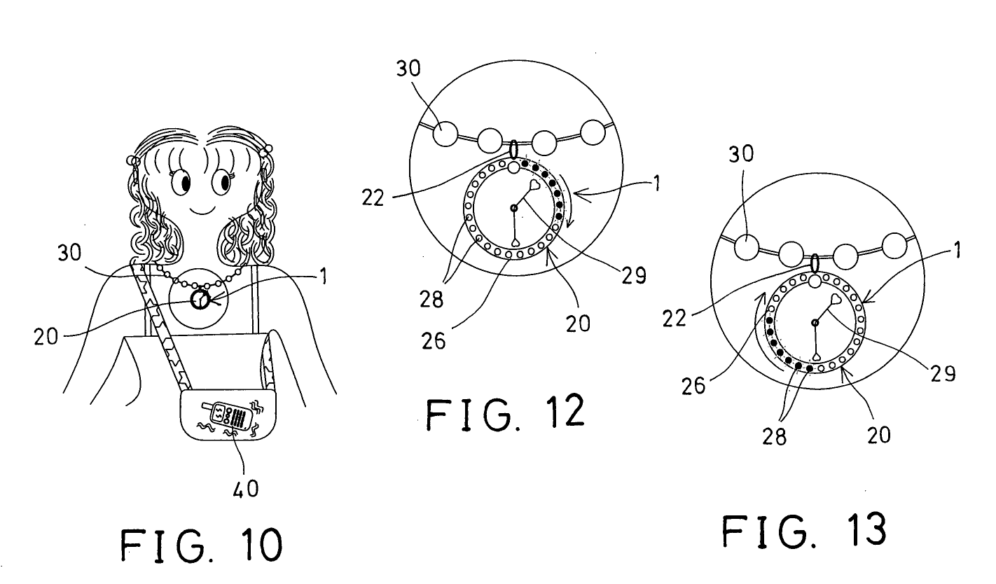

Alternatively, without the fastening belt 10, as shown in FIGS. 10-13, the watch member 20 may also be attached to a necklace 30 with a hook or a snap ring 22 which may include a spring biasing latch 23 to lock the snap ring 22 to the necklace 30, and to prevent the snap ring 22 from being disengaged from the necklace 30, such that the watch member 20 may also be attached to the other portions of the users where the fastening belt 10 may not be applied or fitted onto the users.

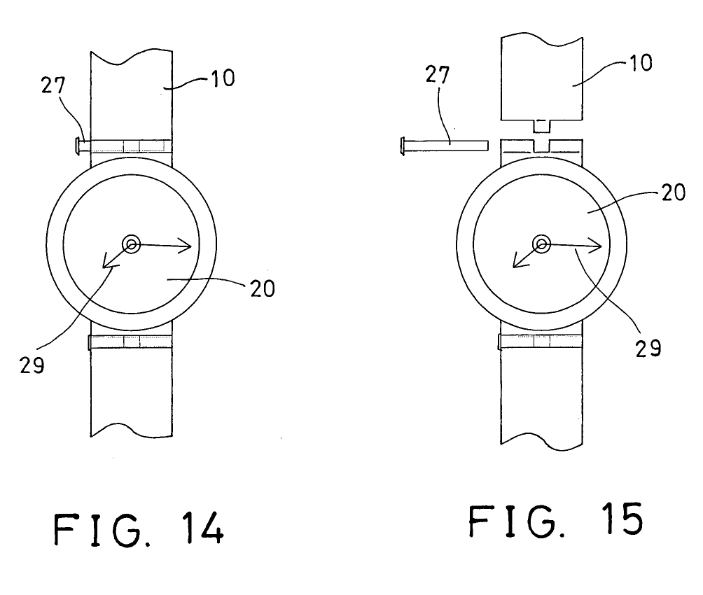

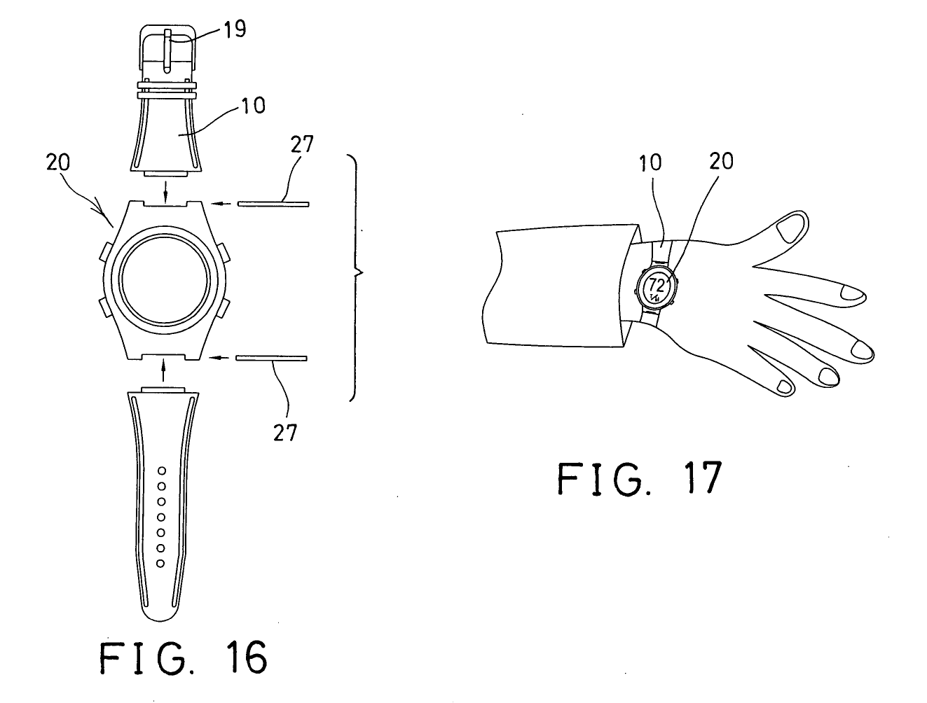

Further alternatively, without the fastening belt 10 and the snap ring 22 and the necklace 30, as shown in FIGS. 14-17, the watch member 20 may also be attached to another fastener belt 10 with one or more pivot pins 27, and the fastener belt 10 may include another lock device 19 for attaching to various portions of users, such as the arms 90, the wrist portion 91 (FIG. 17), the inner portion of the leg 92, or the like.

Referring again to FIGS. 1-2 and 4, the watch member 20 includes an area or a screen 24 to show or display the digital or numerical numbers of the detected heart rate. For example, the normal detected heart rate is around 72 in one minute. The screen 24 may be used to show or display one half of the detected heart rate, or the detected heart rate in one half of a minute (FIGS. 1, 4), or in one minute (FIG. 17).

The watch member 20 includes another area or screen 25 to show or display the status of the detected heart rate. For example, as shown in FIG. 4, when the detected heart rate in one half minute is thirty five (35) that is shown in the screen 24, the screen 25 may have a green color shown or displayed therein, to show that the detected heart rate is within the safe range.

On the contrary, as shown in FIG. 1, when the detected heart rate in one half minute is thirty eight (38) that is shown in the screen 24, the screen 25 may have a red color shown or displayed therein, to show that the detected heart rate is out of the safe range, and thus to warn the users that their heart rates are greater than the normal range.

As shown in FIGS. 1-2, 4, 10, and 12-13, the watch member 20 may further include a further area or screen 26 having a number of light members 28 disposed therein that may be lighted in series, or simultaneously, to show or display that the portable or mobile phone 40 (FIG. 10) has an incoming phone.

As shown in FIGS. 5, 7-10, and 12-15 the watch member 20 may further include two or more pointers 29 to show or display time as that for the typical watches.

Accordingly, the heart rate detecting and displaying device in accordance with the present invention may be attached to various portions of users, and includes a displaying area to show different heart rates with different colors, for allowing the users to easily view the changing of the heart rate.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

1. A heart rate device comprising:
a watch member including a first area to show digit number of heart rates of users,
said watch member including a second area to show status of the heart rates with different colors, and
attaching means for attaching said watch member to the users.

2. The heart rate device as claimed in claim 1, wherein said attaching means includes a fastening belt for attaching to the users, and securing means for securing said watch member to said fastening belt.

3. The heart rate device as claimed in claim 2, wherein and securing means includes a first fastening member attached to said watch member, and a second fastening member attached to said fastening belt and engageable with said first fastening member, to secure said watch member to said fastening belt.

4. The heart rate device as claimed in claim 3, wherein said first and said second fastening members are hook and loop fastening members.

5. The heart rate device as claimed in claim 2, wherein said securing means includes at least one pivot pin to secure said watch member to said fastening belt.

6. The heart rate device as claimed in claim 2, wherein said fastening belt includes a first fastening member and a second fastening member attached thereto and engageable with each other, to attach said fastening belt and thus said watch member to the users.

7. The heart rate device as claimed in claim 1, wherein said watch member includes a third area having a plurality of light members disposed therein.

8. The heart rate device as claimed in claim 1, wherein said watch member includes at least two pointers to show time.

9. The heart rate device as claimed in claim 1, wherein said watch member includes a ring attached thereto.

10. A heart rate device comprising:
a watch member including a first area to show digit number of heart rates of users,
said watch member including a second area having a plurality of light members disposed therein, and
attaching means for attaching said watch member to the users.

11. The heart rate device as claimed in claim 10, wherein said attaching means includes a fastening belt for attaching to the users, and securing means for securing said watch member to said fastening belt.

12. The heart rate device as claimed in claim 11, wherein and securing means includes a first fastening member attached to said watch member, and a second fastening member attached to said fastening belt and engageable with said first fastening member, to secure said watch member to said fastening belt.

13. The heart rate device as claimed in claim 12, wherein said first and said second fastening members are hook and loop fastening members.

14. The heart rate device as claimed in claim 11, wherein said securing means includes at least one pivot pin to secure said watch member to said fastening belt.

15. The heart rate device as claimed in claim 11, wherein said fastening belt includes a first fastening member and a second fastening member attached thereto and engageable with each other, to attach said fastening belt and thus said watch member to the users.

16. The heart rate device as claimed in claim 10, wherein said watch member includes a third area to show status of the heart rates with different colors.

17. The heart rate device as claimed in claim 10, wherein said watch member includes at least two pointers to show time.

18. A heart rate device comprising:
a watch member including a first area to show status of heart rates of users with different colors,
said watch member including a second area having a plurality of light members disposed therein, and
attaching means for attaching said watch member to the users.

19. The heart rate device as claimed in claim 18, wherein said watch member includes at least two pointers to show time.
